(19) United States
(12) Patent Application Publication
(10) Pub. No.: US 2025/0084123 A1
(43) Pub. Date: Mar. 13, 2025

(54) MULTIPRODUCT RESIN REUSE (MRR) FOR THE DEVELOPMENT AND CLINICAL MANUFACTURE OF THERAPEUTIC PROTEINS

(71) Applicant: Merck Sharp & Dohme LLC, Rahway, NJ (US)

(72) Inventors: Collette M. Cutler, Bloomingdale, NJ (US); Hong Li, Basking Ridge, NJ (US); Ren Liu, Walnut Creek, CA (US); Jeffrey T. McPhee, Cazenovia, NY (US); Douglas Dennis Richardson, II, Collegeville, PA (US); Patricia A. Rose, Bridgewater, NJ (US); Patricia Rowicki, Warren, NJ (US)

(73) Assignee: Merck Sharp & Dohme LLC, Rahway, NJ (US)

(21) Appl. No.: 18/798,969

(22) Filed: Aug. 9, 2024

Related U.S. Application Data
(60) Provisional application No. 63/519,374, filed on Aug. 14, 2023.

(51) Int. Cl. C07K 1/22; C07K 16/00

(52) U.S. Cl. CPC C07K 1/22; C07K 16/00

(57) ABSTRACT

The present invention provides a method for multiproduct resin reuse (MRR) utilizing ionic exchange chromatography columns. The methods of the invention outline the cleaning steps necessary for reuse as well as the analytical processes used in determining product carryover from production of one product to the next. The invention outlines a successful MRR strategy that can be utilized not just in lab-scale studies, but in a GMP manufacturing setting.

### CROSS-REFERENCE TO RELATED APPLICATIONS

This application claims the benefit of U.S. Provisional Patent Application Ser. No. 63/519,374 filed Aug. 14, 2023, the entire contents of which are incorporated by reference herein.

### FIELD

This disclosure relates generally to chromatography methods. In particular, this disclosure relates to methods of purifying multiple distinct therapeutic products using reused chromatography resins and media.

### BACKGROUND

Protein therapeutics, such as monoclonal antibodies (mAbs) or Fc fusion proteins, are typically purified through a combination of chromatographic and filtration unit operations. Chromatography remains a critical and widely used purification technology due to its high resolution (Carta G., Jungbauer A. Protein Chromatography. Wiley-VCH Verlag GmbH & Co. KGaA; 2010. Downstream processing of biotechnology products; pp. 1-55; Walter J., Gottschalk U. Concepts for disposables in biopharmaceutical manufacture. In: Shire S. J., Gombotz W., Bechtold-Peters K., Andya J., editors. Current Trends in Monoclonal Antibody Development and Manufacturing. Springer; New York: 2010. pp. 87-99). Most purification processes necessitate the use of Protein-A based affinity chromatography. The advantage to the use of Protein-A based affinity chromatography are high recovery yields and production of a highly pure product in a single step. Nevertheless, additional chromatography steps are usually employed as polishing steps. One such methodology involves the use of ionic exchange resins, such as anion exchange (AEX) and cation exchange (CEX) chromatography, although other modes of chromatography can be chosen as appropriate. These additional steps utilizing such resins result in increased viral, host cell protein, and DNA clearance, as well as the removal of aggregates, unwanted product variant species, and other minor contaminants (Murphy C, Devine T, O'Kennedy R. Technology advancements in antibody purification. Antibody Technology Journal; 2016, 6:17-32).

However, chromatography resins used for the purification of biopharmaceuticals are generally dedicated for use in the production of a single product. For clinical manufacturing, and in good manufacturing practice (GMP) settings, this can result in a resin being used for only a fraction of its potential lifetime. Lab- and small-scale validated resin reuse studies have shown that resins can be cycled up to 100-200 times before the product quality or process performance show any signs of declination. This represents a column lifetime that is greater than ten times the number of cycles required to support the manufacturing needs of early clinical development of a biopharmaceutical product.

A multiproduct resin reuse (MRR) methodology has been previously described by Sharnez et al. (Sharnez R et al., 2018 “Multiproduct Resin Reuse for Clinical and Commercial Manufacturing-Methodology and Acceptance Criteria”. PDA J Pharm Sci and Tech, 72:584-598). However, although MRR methodologies have been successfully implemented on lab scale and pilot plant scale (Sharnez et al., 2018; Mahajan, E., Werber, J., Kothary, K., Larson, T. One resin, multiple products: A green approach to purification. In: Developments in Biotechnology and Bioprocessing. American Chemical Society. ACS Symposium Series; 2013, 1125:87-111), no published work exists regarding the use of an MRR approach in the production of biologics in a GMP environment. Therefore, a need exists for extending the use of resins to multiple products which in turn can significantly reduce resin waste and cost. Furthermore, establishing a successful resin reuse protocol can improve manufacturing flexibility during periods of raw material shortage, as occurred throughout the COVID pandemic.

The methods described herein provide detailed examples of the use of an MRR strategy in the GMP manufacturing of an antibody to support First-In-Human (FIH) studies. Specifically, an AEX and CEX MRR strategy is described. Clearance of carryover biological product generated through the production of one product is demonstrated by cleaning the AEX and CEX manufacturing columns with sodium hydroxide to ensure inactivation and degradation of the carryover protein. This is followed by a blank buffer elution that is tested using various analytical methodologies to ensure reduction of the carryover protein to an acceptable level, before the columns are reused in the production of a second product.

Thus, the procedure disclosed herein represents a viable and novel methodology for the reuse of ionic exchange resins in GMP manufacturing of biologics which maximizes column lifetime, without compromising product quality.

### SUMMARY

The present disclosure provides methods for preparing chromatography materials used in the manufacturing stream of a first polypeptide for reuse in the manufacturing stream of a subsequent polypeptide. In certain embodiments, the methods provided are suitable for use in the GMP scale manufacturing of biopharmaceutical products.

In one aspect, the present disclosure provides a method to prepare chromatography material used in the purification of a first polypeptide for reuse in the purification of a second polypeptide, the method comprising the steps of: a) obtaining the chromatography material used in the purification of the first polypeptide, optionally wherein the first polypeptide has been eluted from the chromatography material; b) passing one or more material volumes of regeneration buffer through the chromatography material, wherein the regeneration buffer comprises from about 0.05 M to about 2.0 M NaOH and a pH from about pH 12.0 to about pH 14.0; c) incubating the chromatography material from about 0° C. to about 32° C. for at least from about 10 minutes to at least about 180 minutes in the presence of at least one of the one or more material volumes of regeneration buffer; d) adding one or more material volumes of elution buffer to the chromatography material, wherein the elution buffer is formulated for use with the chromatography material and the first polypeptide; e) optionally collecting the one or more material volumes of elution buffer from the chromatography material eluate, and optionally testing the one or more material volumes for the presence of the first polypeptide; and f) equilibrating the chromatography material for reuse with the second polypeptide.

In some embodiments the first and second polypeptides are the same. In other embodiments, the first and second polypeptides are different. In preferred embodiments, the properties of the first and second polypeptides and/or one or more of their manufacturing processes are the same, similar, or compatible. In specific embodiments, the properties of the first and second polypeptides and/or the manufacturing processes that are the same, similar, or compatible are selected from the group consisting of: pI, IgG subtype, molecule-type, manufacturing cell line, upstream process, and downstream process.

In some embodiments, the first and second polypeptides are selected from the group consisting of: an enzyme; a hormone; a fusion protein; an Fc-containing protein; an immunoconjugate; a cytokine; and an antibody or antigen binding fragment thereof.

In some embodiments, the antibody is selected from the group consisting of: a monoclonal antibody; a chimeric antibody; a humanized antibody; a human antibody; and a multispecific antibody.

In some embodiments, the multispecific antibody is a bispecific antibody or a trispecific antibody.

In some embodiments, the antigen binding fragment thereof is selected from the group consisting of: a Fab fragment; a Fab′ fragment; a F(ab′)2 fragment; an scFv; a di-scFv; a bi-scFv; a tandem (di, tri) scFv; an Fv; a sdAb; a tri-functional antibody; a BiTE; a diabody; and a triabody.

In some embodiments, the first polypeptide has been eluted from the chromatography material in step (a). In other embodiments, the first polypeptide has not been eluted from the chromatography material in step (a).

In some embodiments, the chromatography material is composed of one chromatography material. In some embodiments, the chromatography material can be made up of one or more than one chromatography material (e.g., a first and/or second chromatography material). In some embodiments, the chromatography material comprises first and second chromatography materials. In some embodiments, the first and second chromatography materials are operated discontinuously in series. In other embodiments, the first and second chromatography materials are operated continuously in series.

In some embodiments, one material volume of regeneration buffer is passed through the chromatography material. In some embodiments, two material volumes of regeneration buffer are passed through the chromatography material. In some embodiments, three material volumes of regeneration buffer are passed through the chromatography material. In some embodiments, four material volumes of regeneration buffer are passed through the chromatography material. In some embodiments, five material volumes of regeneration buffer are passed through the chromatography material. In some embodiments, more than five material volumes of regeneration buffer are passed through the chromatography material.

In some embodiments, the regeneration buffer comprises about 0.05 M NaOH and a pH at about pH 13.0. In some embodiments, the regeneration buffer comprises about 0.1 M NaOH and a pH at about pH 13.0. In some embodiments, the regeneration buffer comprises about 0.5 M NaOH and a pH at about pH 13.0. In some embodiments, the regeneration buffer comprises about 1.0 M NaOH and a pH at about pH 13.0. In some embodiments, the regeneration buffer comprises about 1.5 M NaOH and a pH at about pH 13.0. In some embodiments, the regeneration buffer comprises about 2.0 M NaOH and a pH at about pH 13.0.

In some embodiments, the chromatography material is incubated at from about 20° C. to about 22° C. for at least about 10 minutes in the presence of at least one of the one or more material volumes of regeneration buffer. In some embodiments, the chromatography material is incubated at from about 20° C. to about 22° C. for at least about 20 minutes in the presence of at least one of the one or more material volumes of regeneration buffer. In some embodiments, the chromatography material is incubated at from about 20° C. to about 22° C. for at least about 40 minutes in the presence of at least one of the one or more material volumes of regeneration buffer. In some embodiments, the chromatography material is incubated at from about 20° C. to about 22° C. for at least about 60 minutes in the presence of at least one of the one or more material volumes of regeneration buffer. In some embodiments, the chromatography material is incubated at from about 20° C. to about 22° C. for at least about 120 minutes in the presence of at least one of the one or more material volumes of regeneration buffer. In some embodiments, the chromatography material is incubated at from about 20° C. to about 22° C. for at least about 180 minutes in the presence of at least one of the one or more material volumes of regeneration buffer.

In some embodiments, the chromatography material is selected from the group consisting of: an affinity chromatography material; an ion exchange chromatography material; a monolith chromatography material; a hydrophobic interaction chromatography material; and a mixed mode chromatography material.

In some embodiments, the affinity chromatography material is a protein A affinity chromatography material. In some embodiments, the protein A affinity chromatography material is selected from the group consisting of: MAbSelect; MAbSelect SuRe; and MAbSelect SuRe LX.

In some embodiments, the ion exchange chromatography material is an anion exchange chromatography (AEX) material. In some embodiments, the AEX material is POROS 50HQ.

In other embodiments, the ion exchange chromatography material is a cation exchange chromatography (CEX) material. In some embodiments, the CEX material is Poros 50HS.

In some embodiments, the elution buffer comprises acetic acid and/or from about 0.01 M to about 0.5 M sodium acetate at a pH of from about pH 2.5 to about pH 5.0.

In some embodiments, optionally testing the one or more material volumes of elution buffer for the presence of the first polypeptide comprises one or more analytical assay methods selected from the group consisting of: non-reduced sodium dodecyl sulfate-polyacrylamide gel electrophoresis (SDS PAGE); ultra-performance liquid chromatography-size exclusion chromatography (UPLC-SEC); intact mass liquid chromatography-mass spectrometry (IM LC-MS); reduced peptide mapping (RPM); multiple reaction monitoring-liquid chromatography (LC-MRM); Micro BCA; residual binding enzyme-linked immunosorbent assay (ELISA); and total organic carbon (TOC).

In some embodiments, the first polypeptide is present in the one or more material volumes of elution buffer below a level of quantitation (LOQ) for the one or more analytical assay methods selected.

In some embodiments, the first polypeptide is present in the one or more material volumes of elution buffer as one or more protein fragments, and the total amount of polypeptide fragments are below an acceptable limit for a reference purity. In some embodiments, the reference impurity is gelatin. In some embodiments, the acceptable limit is 650 μg/dose.

In some embodiments, the chromatography material is linked continuously or discontinuously to one or more preceding or subsequent chromatography materials.

In some embodiments, the reused chromatography material is used to manufacture the second polypeptide at large scale. In some embodiments, the large scale is a GMP scale.

In another aspect, the present disclosure provides a method to prepare chromatography material used in the purification of a first polypeptide for reuse in the purification of a second polypeptide, the method comprising the steps of:
a) obtaining the chromatography material used in the purification of the first polypeptide, optionally wherein the first polypeptide has been eluted from the chromatography material;
b) passing one or more material volumes of regeneration buffer through the chromatography material, wherein the regeneration buffer comprises about 1.0 M NaOH at a pH of about pH 13.0;
c) incubating the chromatography material at from about 20° C. to about 22° C. for about 20 minutes in the presence of at least one of the one or more material volumes of regeneration buffer;
d) adding one or more material volumes of elution buffer to the chromatography material, wherein the elution buffer comprises from about 0.01 M to about 0.5 M sodium acetate at a pH of from about pH 2.5 to about pH 5.0;
e) optionally collecting the one or more material volumes of elution buffer from the chromatography material eluate, and optionally testing the one or more material volumes for the presence of the first polypeptide; and
f) equilibrating the chromatography material for reuse with the second polypeptide.

In some embodiments, the first and second polypeptides have acceptable daily exposure or permitted daily exposure limits more than 10 μg/day.

In some embodiments, the first and second polypeptides have No-Observed-Effect-Level for systemic toxicity of ≥300 mg/kg/week.

In another aspect, the present disclosure provides a polypeptide produced or obtainable by any of the methods disclosed herein.

In some embodiments, the polypeptide is selected from the group consisting of: an enzyme; a hormone; a fusion protein, an Fc-containing protein; an immunoconjugate; a cytokine; and an antibody or antigen binding fragment thereof.

In some embodiments, the antibody is selected from the group consisting of: a monoclonal antibody; a chimeric antibody; a humanized antibody; a human antibody; and a multispecific antibody.

In some embodiments, the multispecific antibody is a bispecific antibody or a trispecific antibody.

In some embodiments, the antigen binding fragment thereof is selected from the group consisting of: an Fab fragment; an Fab′ fragment; an F(ab′)2 fragment; an scFv; a di-scFv; a bi-scFv; a tandem (di, tri) scFv; an Fv; an sdAb; a tri-functional antibody; a BiTE; a diabody; and a triabody.

In another aspect, the present disclosure provides a composition comprising the polypeptide and a pharmaceutically acceptable carrier. In some embodiments, the composition comprises a therapeutically effective amount of the polypeptide and a pharmaceutically acceptable carrier.

The summary of the technology described above is non-limiting and other features and advantages of the technology will be apparent from the following detailed description, and from the claims.

### BRIEF DESCRIPTION OF THE DRAWINGS

FIG. 1 is a schematic depiction of an MRR strategic work-flow for GMP manufacturing.

FIG. 2 is a schematic depiction of assessments for deciding whether to proceed with MRR.

FIG. 3 depicts a decision tree to assess risk and decide whether an MRR strategy is appropriate to implement in the manufacturing of a biopharmaceutical product.

FIG. 4 is a schematic depiction of the chromatography purification process for a Product A and Product B.

FIG. 5 is a sodium dodecyl sulfate-polyacrylamide gel electrophoresis (SDS-PAGE) gel image which demonstrates that Product A was effectively degraded after incubation with 1 M NaOH at 20° C. for 20 minutes. Lane 1 is 1× non-reducing SDS sample buffer (blank); Lane 2 is a protein molecular weight marker; Lane 3 is 1× non-reducing SDS sample buffer (blank); Lane 4 is Product A drug substance (DS); Lane 5 is Product A DS mixed with deionized H₂O 60 min; Lane 6 is Product A DS treated with 1 M NaOH for 60 min; Lane 7 is 1× non-reducing SDS sample buffer (blank); Lane 8 is Product A DS; Lane 9 is Product A DS treated with 1 M NaOH for 40 min; Lane 10 is 1× non-reducing SDS sample buffer (blank); Lane 11 is Product A DS.

FIGS. 6A-6D are intact mass liquid chromatography-mass spectrometry (IM LC-MS) chromatograms demonstrating that the intact mass intensity of Product A is undetectable after 20 min incubation with 1 M NaOH. FIG. 6A is a control sample. FIG. 6B is Product A incubated with 1 M NaOH for 20 min. FIG. 6C is Product A incubated with 1 M NaOH for 40 min. FIG. 6D is Product A incubated with 1 M NaOH for 60 min.

FIGS. 7A-7D are ultra-performance size exclusion chromatography (UPLC-SEC) chromatograms demonstrating the purity change of Product A following treatment with 1 M NaOH. FIG. 7A is a control sample. FIG. 7B is Product A incubated with 1 M NaOH for 20 min. FIG. 7C is Product A incubated with 1 M NaOH for 40 min. FIG. 7D is Product A incubated with 1 M NaOH for 60 min.

FIGS. 8A-8F are reduced peptide mapping (RPM) chromatograms demonstrating total ions following treatment of samples with 1 M NaOH. FIG. 8A is a reference sample. FIG. 8B is Product A incubated with 1 M NaOH for 20 min. FIG. 8C is Product A incubated with 1 M NaOH for 40 min. FIG. 8D is Product A incubated with 1 M NaOH for 60 min. FIG. 8E is a control sample. FIG. 8F is a control sample without neutralization.

FIG. 9 is a schematic diagram of the blank run steps and sampling points.

### DETAILED DESCRIPTION

The present disclosure provides methods for preparing chromatography materials used in the manufacturing stream of a first polypeptide for reuse in the manufacturing stream of a subsequent polypeptide. In certain embodiments, the methods provided are suitable for use in the GMP scale manufacturing of biopharmaceutical products.

Certain technical and scientific terms are specifically defined below. Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about,” when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization, and/or use of the substance or composition; through instrumental error in these procedures; through differences in the manufacture, source, or purity of the ingredients employed to make or use the compositions or carry out the procedures; and the like. In certain embodiments, “about” can mean a variation of +0.1%, 0.5%, 1%, 2%, 3%, 4%, 5%, or 10% of the value.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

As used herein, the term “same” refers to polypeptides or purification processes which are identical or indistinct. For example, polypeptides having identical amino acid sequences are the same, whereas purification processes employing identical steps, chromatography materials, buffers, and conditions are the same. As used herein, the term “similar” refers to non-identical polypeptides which have comparable product characteristics (e.g., pI, modality, and mechanism of action (MoA)), whereas similar purification processes may differ in one or more step, chromatography material, buffer, or condition. Despite being non-identical, similar products are “compatible,” as defined herein, when their respective manufacturing processes (e.g., cell lines, cell culture media and conditions, buffers, separation mechanisms between products and impurities, etc.) are functionally interchangeable or can be manipulated or modified to be functionally interchangeable.

The term “condition,” “operating condition,” “operation condition,” “processing condition,” or “process condition,” as used exchangeably herein, refers to the condition for operating a chromatographic process. The condition can be an equilibration condition, loading condition, wash condition, regeneration condition, elution condition, and/or storage condition, etc. The condition includes but is not limited to the type of the chromatographic resin, the resin backbone, the resin ligand, the pH of the operating solution, the composition of the operating buffer solution, the concentration of each ingredient of the operating buffer solution, the conductivity of the operating buffer solution, the ionic strength of the operating solution, the cationic strength of the operating solution, the anionic strength of the operating buffer solution, or a combination of two or more above factors.

The term “buffer” refers to the solution used in operating a chromatographic process. The buffer solution can be an equilibration buffer, loading or feed buffer, wash buffer, regeneration buffer, elution solution, and/or storage solution, etc.

“Eluate,” as used herein, refers to the liquid that passes through a chromatography material. In some embodiments, the eluate is the flowthrough of a loading solution. In other embodiments, the eluate comprises the elution solution that passes through the chromatography material and any additional components eluted from the chromatography material.

The phrase “in series” as used herein with regard to chromatography refers to having a first chromatography step followed by a second chromatography step. Additional steps and chromatography materials may be included between or after the first chromatography step and the second chromatography step.

The term “continuous” as used herein with regard to chromatography refers to having more than one chromatography material (e.g., a first chromatography material and a second chromatography material) either directly connected or connected through some other mechanism which allows for continuous flow between the two chromatography materials. The term “discontinuous” as used herein with regard to chromatography refers to having more than one chromatography material (e.g., a first chromatography material and a second chromatography material) which are separated, e.g., by one or more intervening steps, chromatography materials, or mechanism, such that the flow between the more than one chromatography materials is sequentially interrupted.

The term “polypeptide” refers to polymers of amino acids of any length. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The term “polypeptide” as used herein specifically encompasses antibodies.

“Purified” polypeptide (e.g., a purified antibody) means that the polypeptide has been increased in purity, such that it exists in a form that is more pure than it exists in its natural environment and/or when initially synthesized and/or amplified under laboratory conditions. Purity is a relative term and does not necessarily mean absolute purity.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific and trispecific antibodies), humanized, fully human antibodies, chimeric antibodies, and camelized single domain antibodies.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional, bispecific, trifunctional, and trispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies; and multispecific antibodies formed from antibody fragments.

“Monoclonal antibody,” or “mAb,” or “Mab,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The term “isolated” as used herein refers to a molecule that has been separated from at least some of the components with which it is typically found in nature or produced. For example, a polypeptide (e.g., an antibody) is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated.”

The term “pharmaceutically acceptable” as used herein refers to a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes but is not limited to such sterile liquids as water and oils. The characteristics of the carrier will depend on the route of administration.

The term “therapeutically effective amount” refers to a quantity of a specific substance sufficient to achieve a desired effect in an individual being treated. For instance, this may be the amount necessary to alleviate any particular disease symptom or inhibit or reduce the severity of a disease or disorder in an individual.

### Methods of Multiproduct Resin Reuse

The present disclosure provides methods for preparing chromatography materials used in the manufacturing stream of a first polypeptide for reuse in the manufacturing stream of a subsequent polypeptide. In certain embodiments, the methods provided are suitable for use in the GMP scale manufacturing of biopharmaceutical products.

In one aspect, the present disclosure provides a method to prepare chromatography material used in the purification of a first polypeptide for reuse in the purification of a second polypeptide, the method comprising the steps of: a) obtaining the chromatography material used in the purification of the first polypeptide, optionally wherein the first polypeptide has been eluted from the chromatography material; b) passing one or more material volumes of regeneration buffer through the chromatography material, wherein the regeneration buffer comprises from about 0.05 M to about 2.0 M NaOH and a pH from about pH 12.0 to about pH 14.0; c) incubating the chromatography material at from about 0° C. to about 32° C. for at least from about 10 minutes to at least about 180 minutes in the presence of at least one of the one or more material volumes of regeneration buffer; d) adding one or more material volumes of elution buffer to the chromatography material, wherein the elution buffer is formulated for use with the chromatography material and the first polypeptide; e) optionally collecting the one or more material volumes of elution buffer from the chromatography material eluate, and optionally testing the one or more material volumes for the presence of the first polypeptide; and f) equilibrating the chromatography material for reuse with the second polypeptide.

In some embodiments the first and second polypeptides are the same. In other embodiments, the first and second polypeptides are different.

In some embodiments, the first and second polypeptides are selected from the group consisting of: an enzyme; a hormone; a fusion protein, an Fc-containing protein; an immunoconjugate; a cytokine; and an antibody or antigen binding fragment thereof.

In some embodiments, the antibody is selected from the group consisting of: a monoclonal antibody; a chimeric antibody; a humanized antibody; a human antibody; and a multispecific antibody.

In some embodiments, the multispecific antibody is a bispecific antibody or a trispecific antibody.

In some embodiments, the antigen binding fragment thereof is selected from the group consisting of: a Fab fragment; a Fab′ fragment; a F(ab′)2 fragment; an scFv; a di-scFv; a bi-scFv; a tandem (di, tri) scFv; an Fv; an sdAb; a tri-functional antibody; a BiTE; a diabody; and a triabody.

The invention provides buffers for use in the methods of the invention. Elution buffers are generally used to remove a material from a chromatography material; e.g., a desired material or an undesired material such as a contaminant. Both gradient elution and isocratic elution belong to the elution options. During isocratic elution, the mobile phase composition remains constant throughout the procedure. In contrast, during gradient elution the composition of the mobile phase is altered during the elution process.

In some embodiments, the first polypeptide has been eluted from the chromatography material in step (a). In other embodiments, the first polypeptide has not been eluted from the chromatography material in step (a).

In some embodiments, the elution buffer comprises acetic acid. In some embodiments, the elution buffer comprises sodium acetate. In some embodiments, the elution buffer comprises from about 0.01 M to about 0.5 M sodium acetate and a pH from about pH 2.5 to about pH 5.0. In some embodiments, the elution buffer comprises about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 M sodium acetate and a pH from about pH 2.5 to about pH 5.0.

A regeneration buffer is generally used to recharge a column following a chromatography procedure. In particular embodiments, a regeneration buffer inactivates or degrades a first polypeptide remaining on a column after a first purification stream prior to reusing the column for the purification of a second polypeptide. In some embodiments, the regeneration buffer comprises from about 0.05 M to about 2.0 M NaOH and a pH from about pH 12.0 to about pH 14.0. In some embodiments, the regeneration buffer comprises about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.0 M NaOH and a pH from about pH 12.0 to about pH 14.0. In certain exemplary embodiments, the regeneration buffer comprises about 1.0 M NaOH and a pH at about pH 13.0.

In some embodiments, one material volume of regeneration buffer is passed through the chromatography material. In some embodiments, two material volumes of regeneration buffer are passed through the chromatography material. In some embodiments, three material volumes of regeneration buffer are passed through the chromatography material. In some embodiments, four material volumes of regeneration buffer are passed through the chromatography material. In some embodiments, five material volumes of regeneration buffer are passed through the chromatography material. In some embodiments, more than five material volumes of regeneration buffer are passed through the chromatography material.

In some embodiments, the chromatography material is incubated at from about 20° C. to about 22° C. for at least from about 10 to at least about 180 minutes in the presence of at least one of the one or more material volumes of regeneration buffer. In some embodiments, the chromatography material is incubated at from about 20° C. to about 22° C. for at least about 10, 11, 12, 13, 13, 14, 15, 16, 17, 17, 18, 19, 20, 21, 21, 22, 23, 24, 25, 25, 26, 27, 28, 29, 29, 30, 31, 32, 33, 33, 34, 35, 36, 37, 37, 38, 39, 40, 41, 41, 42, 43, 44, 45, 45, 46, 47, 48, 49, 49, 50, 51, 52, 53, 53, 54, 55, 56, 57, 57, 58, 59, 60, 61, 61, 62, 63, 64, 65, 65, 66, 67, 68, 69, 69, 70, 71, 72, 73, 73, 74, 75, 76, 77, 77, 78, 79, 80, 81, 81, 82, 83, 84, 85, 85, 86, 87, 88, 89, 89, 90, 91, 92, 93, 93, 94, 95, 96, 97, 97, 98, 99, 100, 101, 101, 102, 103, 104, 105, 105, 106, 107, 108, 109, 109, 110, 111, 112, 113, 113, 114, 115, 116, 117, 117, 118, 119, 120, 121, 121, 122, 123, 124, 125, 125, 126, 127, 128, 129, 129, 130, 131, 132, 133, 133, 134, 135, 136, 137, 137, 138, 139, 140, 141, 141, 142, 143, 144, 145, 145, 146, 147, 148, 149, 149, 150, 151, 152, 153, 153, 154, 155, 156, 157, 157, 158, 159, 160, 161, 161, 162, 163, 164, 165, 165, 166, 167, 168, 169, 169, 170, 171, 172, 173, 173, 174, 175, 176, 177, 177, 178, 179, or 180 minutes in the presence of at least one of the one or more material volumes of regeneration buffer. In certain exemplary embodiments, the chromatography material is incubated at from about 20° C. to about 22° C. for about 20 minutes in the presence of at least one of the one or more material volumes of regeneration buffer.

Equilibration of the exchanger may be accomplished by flowing an equilibration buffer through the exchanger to establish the appropriate pH, conductivity, concentration of salts, etc. In some embodiments, the equilibration buffer may include any of a wide range of options depending on the binding requirements of a particular protein. The equilibration buffer will normally include a buffering compound to confer adequate pH control. Buffering compounds may include but are not limited to MES, HEPES, BICINE, imidazole, Tris, phosphate such as PBS, citrate, or acetate, or some mixture of the foregoing, or other buffers. The concentration of a buffering compound in an equilibration buffer commonly ranges from 20 to 100 mM depending of the protein of interest. The pH of the equilibration buffer may range from about pH 4.0 to pH 9.5, more preferably 6 to 8. The equilibration buffer may also comprise a salt to adjust ionic strength or conductivity of the solution as needed. Examples of suitable salts include ammonium sulfate, sodium sulfate, potassium sulfate, ammonium phosphate, sodium phosphate, potassium phosphate, potassium chloride, sodium chloride, or mixtures thereof. A nonlimiting example of an equilibration buffer is about 25 mM Tris, and about 25 mM NaCl and a pH at about pH 7.1. A storage buffer is generally used to maintain a chromatography material when not in use; for example, with a microcode to prevent contamination. A nonlimiting example of a storage buffer is about 100 mM sodium acetate, about 2% benzyl alcohol, and at about pH 5.0.

In some embodiments, the method comprises first and second chromatography materials. In some embodiments, the first and second chromatography materials are operated discontinuously in series. In other embodiments, the first and second chromatography materials are operated continuously in series. In some embodiments, the chromatography material is linked continuously or discontinuously to one or more preceding or subsequent chromatography materials.

In some embodiments, the chromatography material is selected from the group consisting of: an affinity chromatography material; an ion exchange chromatography material; a monolith chromatography material; a hydrophobic interaction chromatography material; and a mixed mode chromatography material.

In some embodiments, the chromatography material is an affinity chromatography material. Examples of affinity chromatography materials include, but are not limited to chromatography materials derivatized with protein A or protein G. Examples of affinity chromatography material include, but are not limited to, Prosep-VA, Prosep-VA Ultra Plus, Protein A Sepharose fast flow, Tyopearl Protein A, MabSelect™, MabSelect™ SuRe and MabSelect™ SuRe LX. In some embodiments of the above, the affinity chromatography material is an affinity chromatography membrane. In some embodiments, the affinity chromatography material is a Protein G chromatography material. In some embodiments, the affinity chromatography material is a protein A affinity chromatography material. In certain embodiments, the protein A affinity chromatography material is selected from the group consisting of: MabSelect; MabSelect SuRe; and MabSelect SuRe LX.

In some embodiments, the invention provides methods to regenerate an ion exchange chromatography material for reuse. In some embodiments, the ion exchange material is in a chromatography column. In some embodiments, the chromatography column is used for large-scale, e.g., GMP manufacturing-scale, production of a polypeptide product such as an antibody product. In some embodiments, the chromatography material is used to purify multiple antibody products.

In some embodiments, the ion exchange chromatography material is an anion exchange chromatography (AEX) material. In some embodiments, the anion exchange material is in a chromatography column. In some embodiments, the anion exchange chromatography material is a solid phase that is positively charged and has free anions for exchange with anions in an aqueous solution passed over or through the solid phase. In some embodiments of any of the methods described herein, the anion exchange material may be a membrane, a monolith, or resin. In an embodiment, the anion exchange material may be a resin. In some embodiments, the anion exchange material may comprise a primary amine, a secondary amine, a tertiary amine or a quarternary ammonium ion functional group, a polyamine functional group, or a diethylaminoaethyl functional group. In some embodiments of the above, the anion exchange chromatography material is an anion exchange chromatography material. In some embodiments of the above, the anion exchange chromatography material is an anion exchange chromatography membrane. In some embodiments, the anion exchange chromatography material is used for large-scale, e.g., GMP manufacturing-scale, production of a polypeptide product such as an antibody product. Examples of AEX materials are known in the art and include, but are not limited to Poros HQ50, Poros PI50, Poros D, Mustang Q, Q Sepharose FF, and DEAE Sepharose. In certain embodiments, the AEX material is POROS 50HQ.

In other embodiments, the ion exchange chromatography material is a cation exchange chromatography (CEX) material. In some embodiments, the cation exchange material is in a chromatography column. In some embodiments, the cation exchange material is a solid phase that is negatively charged and has free cations for exchange with cations in an aqueous solution passed over or through the solid phase. In some embodiments of any of the methods described herein, the cation exchange material may be a membrane, a monolith, or resin. In some embodiments, the cation exchange material may be a resin. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. In some embodiments of the above, the cation exchange chromatography material is a cation exchange chromatography material. In some embodiments of the above, the cation exchange chromatography material is a cation exchange chromatography membrane. In some embodiments of the invention, the chromatography material is not a cation exchange chromatography material. In some embodiments, the cation exchange chromatography material is used for large-scale, e.g., GMP manufacturing-scale, production of a polypeptide product such as an antibody product. Examples of CEX materials are known in the art include, but are not limited to Mustang S, Sartobind S, SO3 Monolith, S Ceramic HyperD, Poros XS, Poros HS50, Poros HS20, SPSFF, SP-Sepharose XL (SPXL), CM Sepharose Fast Flow, Capto S, Fractogel Se HiCap, Fractogel SO3, or Fractogel COO. In certain embodiments, the CEX material is Poros 50HS.

Conditions (e.g., flow rate, material bed height, material bed diameter, buffer formulation and conductivity, etc.) for operating any of the chromatography processes described herein may be based on recommendations of the manufacturer or supplier of the material, as well as standard conditions and variations thereof known in the art.

### Detection of Contaminants and Polypeptide Carryover

“Contaminants” refer to materials that are different from the desired polypeptide product. Contaminants include, without limitation: host cell materials; leached Protein A; nucleic acid; a variant, fragment, aggregate or derivative of the desired polypeptide; another polypeptide (e.g., a polypeptide carried over from a previous purification stream); endotoxin; viral contaminant; cell culture media component, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, and a fungal cell. In certain embodiments, the contaminant is a first polypeptide detected in the eluate of a chromatography material intended for reuse with a second polypeptide.

Art-known detection methods and analytical assay methods may be used to test for the presence of or to quantify polypeptide contaminants. In some embodiments, testing the one or more material volumes of elution buffer for the presence of the first polypeptide comprises one or more analytical assay methods selected from the group consisting of: non-reduced sodium dodecyl sulfate polyacrylamide gel electrophoresis (SDS PAGE); ultra-performance liquid chromatography-size exclusion chromatography (UPLC-SEC); intact mass liquid chromatography-mass spectrometry (IM LC-MS); reduced peptide mapping (RPM); multiple reaction monitoring-liquid chromatography (LC-MRM); Micro BCA; residual binding enzyme-linked immunosorbent assay (ELISA); and total organic carbon (TOC).

In some embodiments, the first polypeptide is present in the one or more material volumes of elution buffer below a level of quantitation (LOQ) for the one or more analytical assay methods selected.

In some embodiments, the first polypeptide is present in the one or more material volumes of elution buffer as one or more protein fragments, and the total amount of polypeptide fragments are below an acceptable limit for a reference purity. In some embodiments, the reference impurity is gelatin. In some embodiments, the acceptable limit is 650 μg/dose.

In some embodiments, the reused chromatography material is used to manufacture the second polypeptide at large scale. In some embodiments, the large scale is a GMP scale.

The following examples are meant to be illustrative and should not be construed as further limiting. The contents of the figures and all references, patents, and published patent applications cited throughout this application are expressly incorporated herein by reference.

### EXAMPLES

### Example 1: MRR Work-Flow for GMP Manufacturing

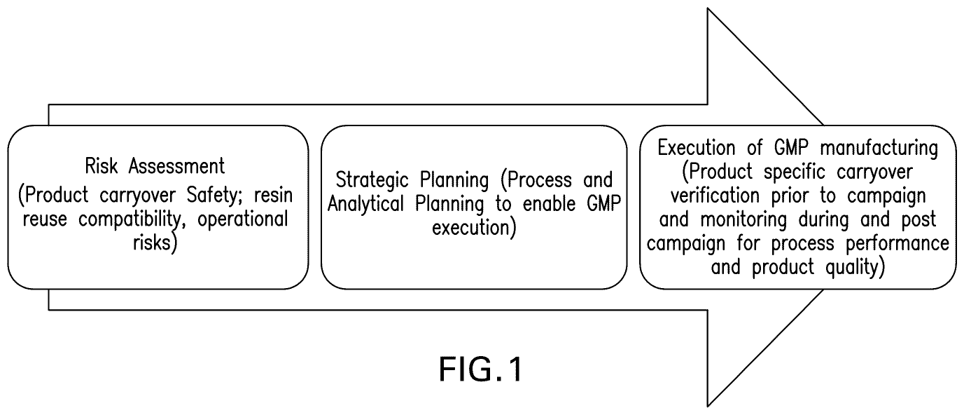

A high-level MRR strategic work-flow for GMP manufacturing is shown in FIG. 1. MRR work-flow development started with a risk assessment that evaluated: (1) the safety impact of a first product carrying over into the manufacturing stream of a subsequent product; and (2) the compatibility of the manufacturing processes of the first and subsequent products. Other risks such as operational and GMP readiness prior to MRR execution were also considered. Next, critical processes and analytical assays were developed. These included: (1) creating an effective cleaning protocol for pilot and GMP scales using small-scale studies; (2) establishing a robust analytical plan to confirm both product inactivation and carryover quantitation meet the established acceptance limits; and (3) executing the MRR strategy in a GMP environment that provided for verification of carryover protein removal from the reused chromatography resin(s) and process performance and product quality monitoring during and after manufacturing.

### Example 2: MRR Risk Assessment

The primary concern when considering an MRR strategy is the safety impact of a first product carrying over into the manufacturing stream of a subsequent product. This risk should be assessed on a product-by-product basis. An evaluation of the properties of a first and subsequent product and their manufacturing processes are important in determining whether these products are compatible for sharing chromatography resins.

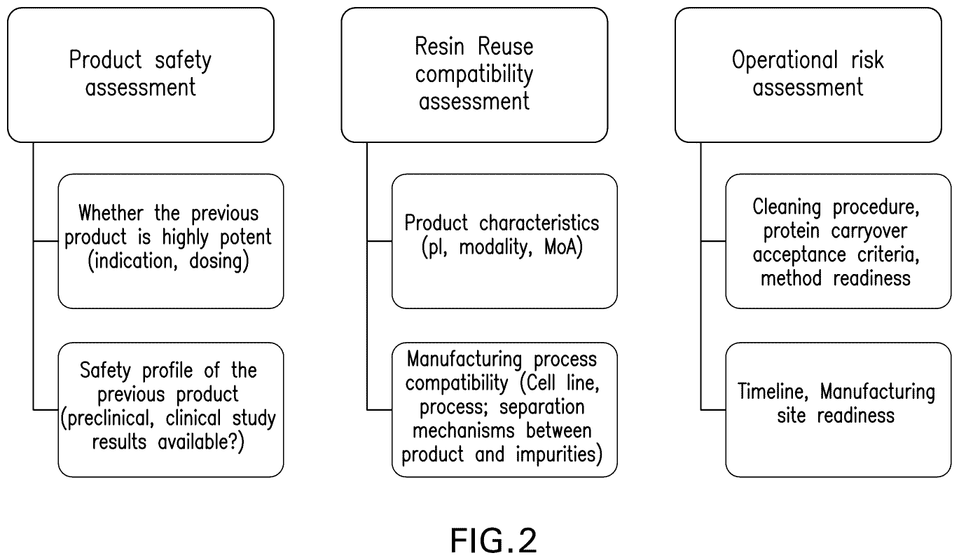

As shown in FIG. 2, various assessments should be performed when deciding whether to proceed with MRR. These include: (1) a product safety assessment, e.g., whether a first product is highly potent (indication, dosing) and has an acceptable preclinical and/or clinical safety profile; (2) a resin reuse safety assessment, e.g., manufacturing process compatibility (cell line, process, separation mechanisms between product and impurities) and product characteristics (pI, modality, MoA); and (3) an operational risk assessment, e.g., cleaning procedure, protein carryover acceptance criteria, method readiness, manufacturing timeline, and manufacturing site readiness.

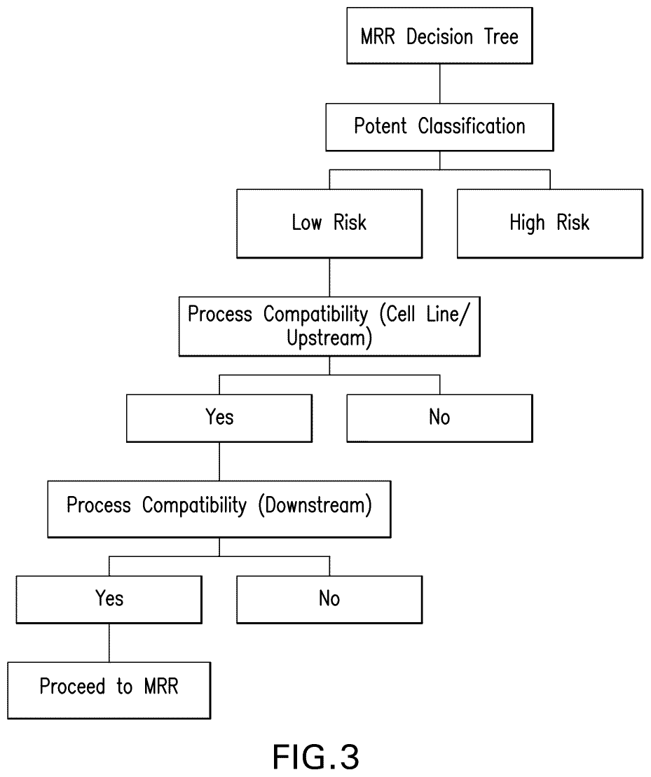

FIG. 3 depicts a decision tree that can be utilized to assess risks when deciding whether an MRR strategy is appropriate for the manufacturing of biopharmaceutical products. The first decision point is the potential risk posed by the carryover of a first product, as biologics are generally specific for their target(s) and can be quite potent. Potent and toxic products with acceptable daily exposure (ADEs) or permitted daily exposure (PDEs) limits less than 10 μg/day are not recommended to be applied for MRR (EMA Q&A 2018, EMA/CHMP/CVMP/SWP/246844/2018, Questions and answers on implementation of risk-based prevention of cross-contamination in production and ‘Guideline on setting health-based exposure limits for use in risk identification in the manufacture of different medicinal products in shared facilities’ (EMA/CHMP/CVMP/SWP/169430/2012)). Due to the low exposure limit of highly potent or toxic products, these may pose a higher safety risk for residual carry-over.

It should be recognized that mechanism of action, the availability of relevant pre-clinical and clinical data, and existing knowledge should be considered for this risk assessment and later used in determining a scientifically justified limit for the target population and route of exposure (Barle E. 2017 Pharmaceutical Technology September 2019. “Using health-based exposure limits to assess risk in cleaning validation”).

### Example 3: MRR Risk Assessment in the GMP Manufacturing of a Biopharmaceutical

“Product A” is a monoclonal antibody and its safety has been evaluated in animal GLP studies, including rats and non-human primates. Overall, Product A was well tolerated and no findings of toxicological significance were observed. A No-Observed-Effect-Level (NOEL) for systemic toxicity was determined to be ≥300 mg/kg/week for Product A. Although no clinical information was available for Product A, the risk of applying an MRR strategy for the manufacture of a subsequent product (i.e., “Product B”) was considered low.

The next crucial assessment when considering the application of MRR was the compatibility of the manufacturing processes for Product A and Product B. The similarities of the product properties (pIs and framework) and the almost identical platform process (cell line, upstream and downstream processes) utilized for both drug substance manufacturing led to the conclusion that process residuals, such as the host cell proteins (HCP) generated in the two processes would be very similar (Table 1).

TABLE 1
Manufacturing Process Compatibility

| | |
|---|---|
| Product A | Product B |

Attribute

| | | |
|---|---|---|
| pI | 8.73 | 7.20 |
| IgG subtype | IgG1 | IgG1 |
| Molecule | Monoclonal | Antibody-based |
| Modality | Antibody | therapeutic |

Manufacturing process (cell line, upstream, downstream)

| | | |
|---|---|---|
| Manufacturing | CHO-K1 | CHO-K1 |
| cell line | | |

| | |
|---|---|
| Upstream process | Fed batch with N-1 perfusion |
| Downstream process | Protein A affinity (MabSelect SuRe), AEX |
| (chromatography | flow through (Poros 50HQ), CEX bind-and- |
| resins) | elute (Poros 50HS) |

Additionally, resin cleaning procedure(s), process controls for removing process residuals by downstream polishing steps, and Product A drug substance release specification were evaluated to ensure product safety. Based on the decision tree, it was decided that an MRR strategy could be utilized in the manufacturing of Product B to support clinical studies.

Operational risks were assessed prior to the actual execution of MRR for Product B GMP manufacturing, which included evaluating cleaning procedures, protein carryover acceptance limits, analytical method readiness, manufacture site readiness, and manufacturing timeline.

### Example 4: Upstream and Downstream Antibody Purification Processes

As indicated in the overall MRR strategy, prior to Product B GMP manufacturing, a quality risk management (QRM) plan based on Product B and its manufacturing process was performed to identify potential Product A cross contamination safety risks, failure modes, detection mechanism, control measures, and residual levels to be accepted. All small- and pilot-scale data, including blank runs, chromatography performances, and quality attribute data were evaluated. Additionally, a protocol driven GMP blank run was also executed, and carryover samples were taken for the Micro BCA and potency ELISA testing. These methods were GMP qualified, and results met the pre-set acceptance criteria.

### Upstream Process

Experiments were carried out using Product A and B process intermediates ranging from harvested cell culture fluid (HCCF) to purified drug substance (DS) (Table 2).

TABLE 2
Process Intermediates Used in MRR

| Intermediate | | |
|---|---|---|
| ID | Definition | Description |
| FNVIP | Filtered Neutralized | Protein A purified HCCF held at |
| | Viral Inactivated | low pH for viral inactivation, |
| | Product | neutralized, and filtered |
| AEXL | Anion Exchange | Intermediate conditioned to pH |
| | Load | and conductivity targets |
| | | (pH 7.0-8.0, conductivity 3.0-6.0 |
| | | mS/cm) for anion exchange |
| | | chromatography load |
| AEXP | Anion Exchange | Product collected from anion |
| | Product | exchange chromatography and |
| | | adjusted to pH 5.0-6.0 |
| CEXL | Cation Exchange | Intermediate conditioned to pH |
| | Load | and conductivity targets |
| | | (pH 5.0-6.0, conductivity 3.0-6.0 |
| | | mS/cm) for cation exchange |
| | | chromatography load |
| CEXP | Cation Exchange | Product collected from cation |
| | Product | exchange chromatography |

Products were produced using proprietary Chinese hamster ovary (CHO) cells in a bioreactor with commercially available media and feeds using established process parameters (Yu M et al., 2011 Biotechnol Bioeng. 2011; 108:1078-1088 “Understanding the intracellular effect of enhanced nutrient feeding toward high titer antibody production process;” Reinhard D et al., 2015 Appl Microbiol Biotechnol. 2015; 99 (11): 4645-4657. “Benchmarking of commercially available CHO cell culture media for antibody production”). Cell culture process conditions including temperature, dissolved oxygen, pH, and agitation were monitored on-line. Glucose, lactate, ammonium, glutamine, glutamate, osmolality, cell growth, and viability were monitored off-line with daily sampling. The final HCCF had a product titer between 3-6 g/L and was stored at 2-8° C. for short term storage or stored at ≤−70° C. for long term prior to processing.

### Downstream Process

Lab-scale chromatography experiments were carried out on an AKTA™ Avant 25 system controlled by UNICORN™ software (Cytiva™, Marlborough, MA, United States). This system has built-in pressure, UV, pH, and conductivity detectors to monitor the chromatography runs. Pilot scale and GMP runs were performed on AKTA™ process skids controlled by UNICORN™ software. Table 3 summarizes the bioreactor scales and column sizes used to perform the purifications of Product A and B defined as lab-, pilot,-and GMP-scale. Column chromatography was run applying the same residence time across scales. Column delta pressure was maintained within acceptable operational parameters.

TABLE 3
Bioreactor Scales and Column Sizes

| | | Column dimensions (ID × Height |
|---|---|---|
| Purification Scale | BRX size (L) | (cm)) |
| Lab-scale ¹ | 3-500 ² | 0.6-2.6 × 20 |
| Pilot-scale | 200 | 14-30 × 15-17 |
| GMP | 2000 | 30-60 × 18-23 |

¹ Lab-scale affinity chromatography experiments used MabSelect SuRe resin while the pilot and GMP production was performed with MabSelect SuRe LX. (The LX resin is based on the same rigid high-flow agarose base matrix and alkali-stabilized ligand as the MabSelect SuRe resin, though can withstand higher NaOH cleaning condition.)
² Material used in the lab-scale purification was from 3-500 L BRX sizes.

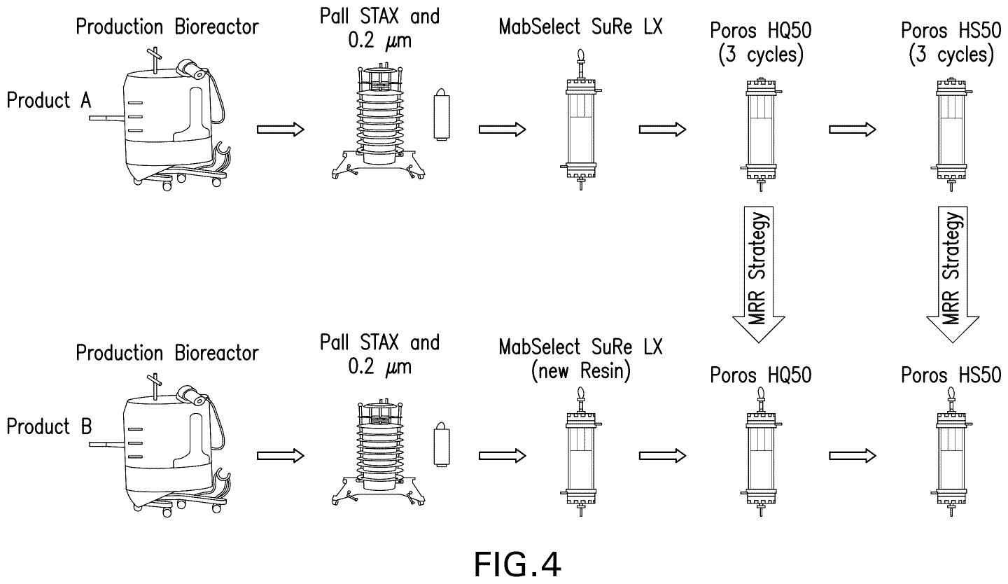

The chromatography purification processes for Product A and Product B are shown in FIG. 4, which is a typical platform for monoclonal antibody purification including affinity capture followed by two polishing chromatography steps. Three cycles of Poros 50HQ and Poros 50HS were run for Product A GMP manufacturing prior to the use for Product B. Fresh MabSelect SuRe LX™ resin was used for Product B GMP manufacturing. The MabSelect Sure™ and MabSelect SuRe LX™ resins were sourced from Cytiva™ (Marlborough, MA, United States). The POROS™ 50HQ and POROS 50HS resins were sourced from Thermo Fisher Scientific Inc™ (Waltham, MA, United States).

Buffer components, including acetic acid, sodium acetate, sodium hydroxide, Tris (hydroxymethyl) aminomethane, monosodium phosphate (supplied as sodium phosphate monohydrate [NaH₂PO4·H₂O]), and disodium phosphate (supplied as sodium phosphate dibasic heptahydrate [Na₂HPO4·7H₂O]) were purchased from Fisher Scientific (Waltham, MA, United States); sodium acetate trihydrate and glacial acetic acid were purchased from EMD Millipore. Tris-HCl was purchased from Mallinckrodt; sodium chloride was purchased from Sigma-Aldrich (Merck KgaA, Darmstadt, Germany). All buffers were between 10 and 50 mM, with sodium chloride salt concentrations between 0-1 M. All buffers were prepared using water for injection or process purified water using ACS grade or better reagents sourced from SigmaAldrich, Thermo Fisher Scientific or similar vendor. All buffers were filtered through a 0.2 μm polyethersulfone (PES) or polyvinylidene fluoride (PVDF) filter prior to use.

### Example 5: Analytical Methods to Evaluate the MRR GMP Process

The analytical methods used to evaluate degradation of Product A, as well as the carryover of Product A in the blank eluate, are shown in Table 4.

TABLE 4
Analytical Methods

| Assay | Description |
|---|---|
| Non-Reduced SDS-PAGE | Degradation of Product A |
| UPLC-SEC | Sized based separation technique to identify |
| | any remaining intact protein |
| Intact Mass (LC-MS) | Evaluation of intact mass to demonstrate |
| | degradation of Product A |
| Reduced Peptide | Identification of degradation pathways for |
| Mapping (LC-MS) | Product A |
| LC-MRM | Quantitation of total protein through |
| | surrogate peptides |
| Micro BCA | Quantitation of total protein after degradation |
| Residual Binding | Specific binding of Product A to demonstrate |
| ELISA | lack of activity for any remaining protein |
| Total Organic Carbon | Quantitation of total organic carbon after |
| (TOC) | water for injection (WFI) rinse |

### Static Hold Degradation Study

A static hold degradation study using NaOH incubation was performed to define the concentration and contact time required to degrade Product A. Table 5 outlines the study design.

TABLE 5
Product A Drug Substance Degradation Study Design

| | | DS | Target DS | | Stock |
|---|---|---|---|---|---|
| | Target NaOH | concentration | Conc (g/L) | | Inactivation |
| Condition | Concentration | (g/L) | after Dilution | Diluent | Solution |
| 1 | 0.1M | 151.5 | 0.4 | WFI | 10M NaOH |
| 2 | 0.5M | | | | |
| 3 | 1.0M | | | | |

Product A drug substance (DS) was diluted to 0.4 mg/mL in deionized purified water (DPW) and incubated for at least 15 min at room temperature (RT), i.e., at from about 20° C. to about 22° C., with gentle mixing. The diluted DS was then spiked with concentrated NaOH solution to reach the experimental target concentration and incubated at room temperature for 20, 40, 60, and 120 min. During the incubation, gentle periodic mixing was completed. At each timepoint the solution was snap frozen. The samples were stored at ≤−70° C. until testing. Following incubation of Product A with 1 M NaOH as described above, the degradation samples were tested to characterize the degradation pathway of the protein and confirm inactivation. Samples were prepared by incubating Product A with 1 M NaOH for 20, 40, and 60 min. Additionally, an untreated control sample was generated to provide a point of reference for the analytical methods used in the study.

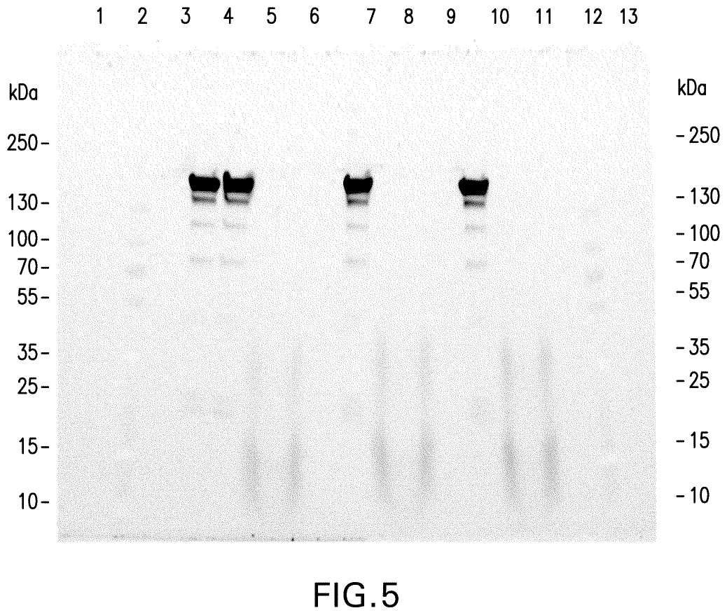

Non-Reduced SDS-PAGE. Caustic treated and nontreated protein samples were analyzed on a gradient (4%-20%) precast polyacrylamide gel stained with a highly sensitive fluorescent stain (SYPRO Ruby). The SDS-PAGE analysis shown in FIG. 5 demonstrates that Product A was effectively degraded after incubation with 1 M NaOH at 20° C. for 20 min. Using a longer incubation time did not significantly increase protein degradation.

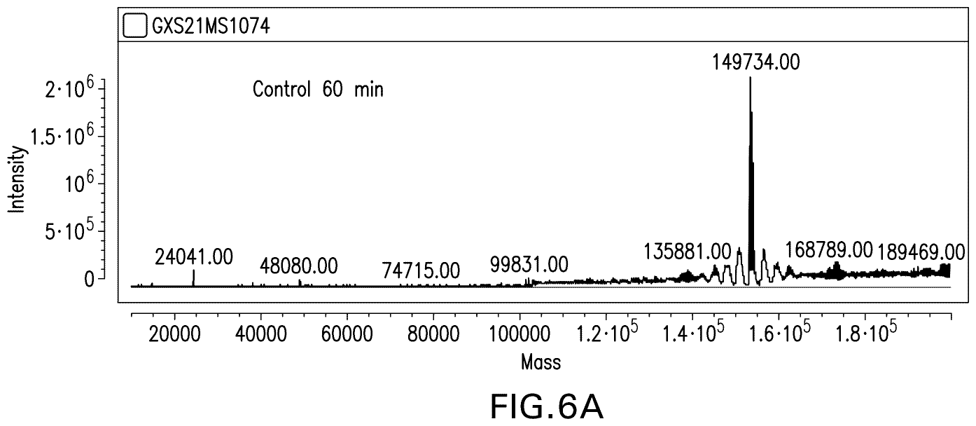

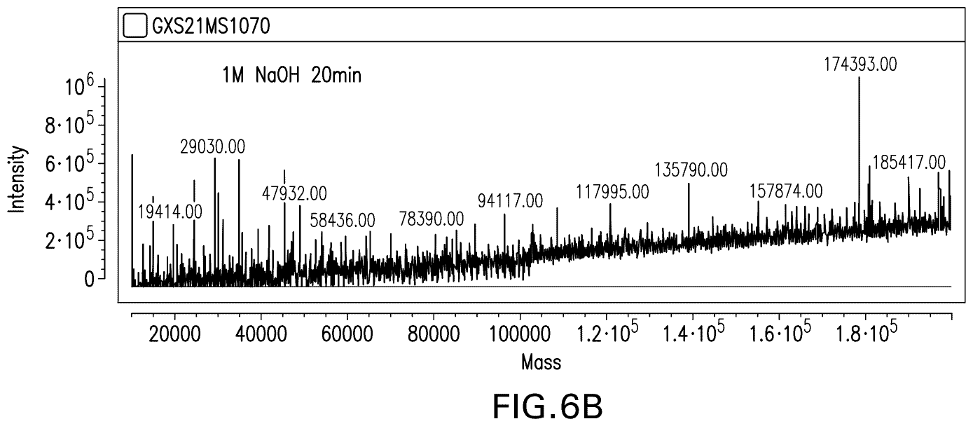

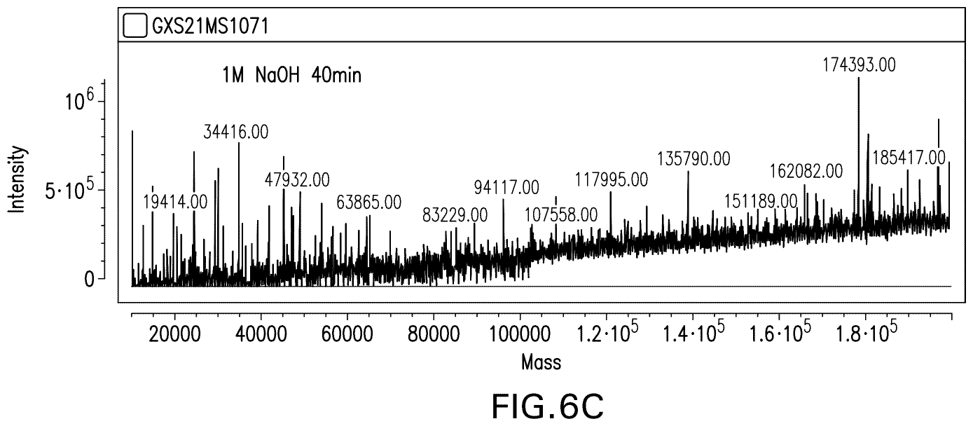

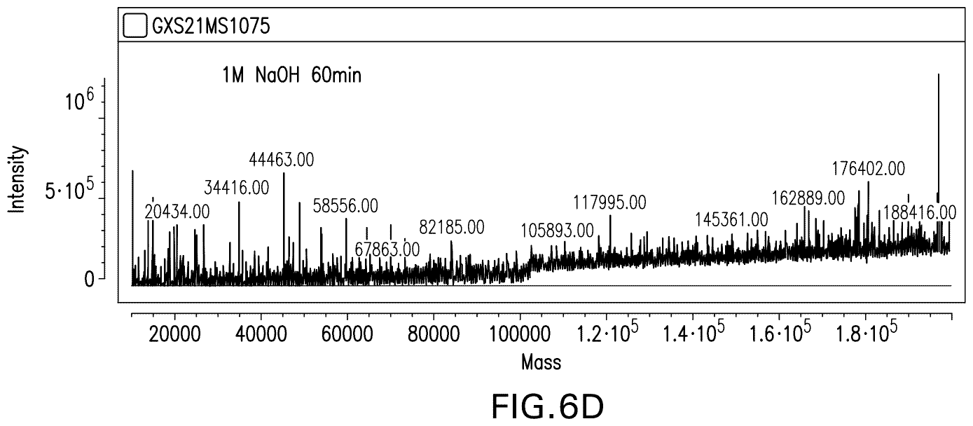

Intact Mass Liquid Chromatography-Mass Spectrometry (IM LC-MS). The samples were analyzed by IM LC-MS using a Waters Xevo G2-XS to analyze the samples. Spectral deconvolution was performed using MaxEnt1 in Expressionist 15.0 (Genedata) using Maximum Entropy Deconvolution algorithm with 20 iterations. FIG. 6B demonstrates that the intact mass intensity of Product A is undetectable after 20 min incubation with 1 M NaOH.

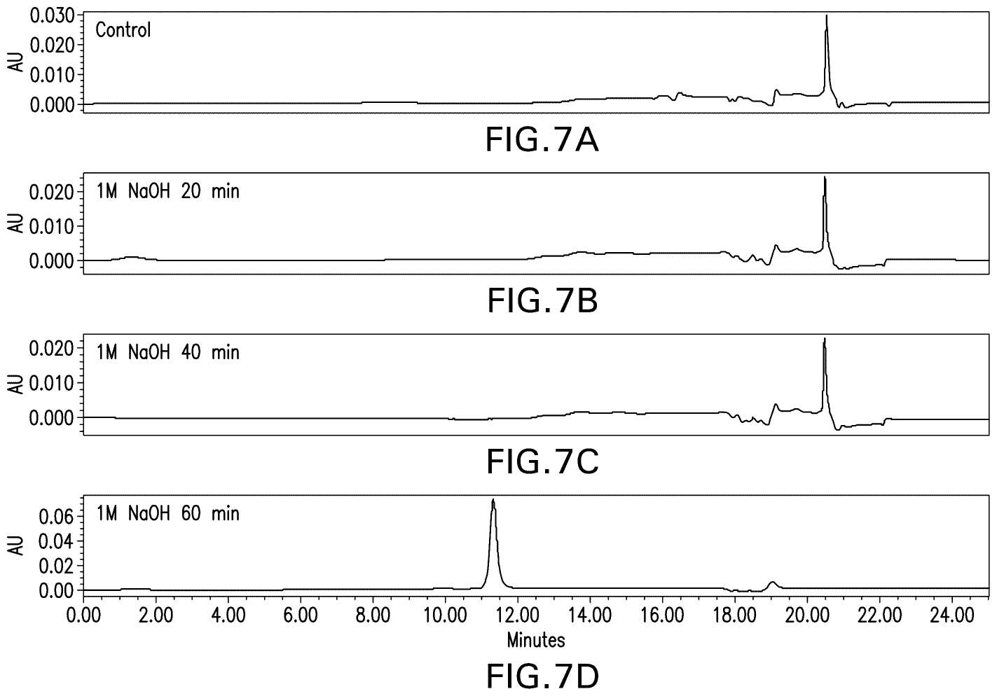

Ultra-Performance Liquid Chromatography-Size Exclusion Chromatography (UPLC-SEC).). The samples were analyzed by UPLC-SEC using a Waters Acquity H-class® liquid chromatography instrument to evaluate the purity change of Product A following treatment with 1 M NaOH. FIG. 7B shows that the monomer peak is no longer present after incubating at 20 mins with 1 M NaOH. Meanwhile, there was an increase in low molecular weight species after incubation with NaOH. The chromatograms suggest that product carryover is essentially eliminated after cleaning the column for 20 min with 1 M NaOH.

Enzyme-Linked Immunosorbent Assay (ELISA). ELISA data was also collected for the degradation samples to understand the level of stress required to confirm inactivation of Product A. The method utilizes LC-MS to confirm the protein degradation pathway relative to unstressed control. This method evaluated the protein following enzymatic digestion and can identify peptides that could be leveraged for focused quantification, in addition to providing information on the extent and nature of the degradation induced by caustic treatment. The standard curve was generated by using Product A reference material in the assay dilution buffer and reported as a unit of ng/mL. The amounts of Product A in the samples were estimated in the quantification range (0.5 ng/mL-200 ng/ml) of the standard curve. Each 1 M NaOH degradation sample was neutralized with 1:1 mix of 0.5 M acetic acid given low performance of ELISA at high pH. Table 6 shows the amount of 0.2 mg/mL Product A samples following incubation with 1 M NaOH for 20 minutes, 40 minutes, and 60 minutes. A stock solution concentration of 0.2 mg/mL was used for Product A to ensure that the working sample concentration was within the range of the method. The results for these samples were all below the limit of quantification (LOQ; 3 ng/mL) and therefore provided evidence of Product A inactivation after incubating with 1 M NaOH for a minimum of 20 minutes.

TABLE 6
ELISA Results of the 1M NaOH Degradation Study

| Sample | Result² |
|---|---|
| 20 min | <LOQ |
| 40 min | <LOQ |
| 60 min | <LOQ |
| Negative Control¹ | NA |

¹Assay dilution buffer treated as a sample.
²LOQ 3 ng/mL.

Reduced Peptide Mapping (RPM). RPM by liquid chromatography mass spectrometry was also performed to measure the degradation of Product A following incubation with 1 M NaOH and subsequent neutralization with 1 M acetic acid over several time points. RPM was performed using a Thermo Q Exactive mass spectrometer to confirm the degradation pathway(s) of Product A relative to an unstressed control. Product A was subjected to enzymatic digestion and subsequently evaluated to identify peptides that could be leveraged for focused quantitation in addition to providing information on the extent and nature of the degradation induced by caustic treatment. Table 7 and Table 8 show the changes in post translation/chemical modification levels on Product A after incubation with 1 M NaOH relative to control. Deamidation of asparagine or glutamine residues were the most common modification with increases of 6-86% upon incubation for 20 minutes relative to the 20 min control. Increases in methionine and tryptophan oxidation and asparagine succinimide were also observed. Importantly, the DTLYITR peptide, which was used for quantitation by LC-MRM, did not show any measurable chemical modification which supports its use as a surrogate peptide for Product A quantitation.

TABLE 7
Post Translation Modifications in Heavy Chain (RPM LC-MS)

| | | Reference | | | | | Control w/o |
|---|---|---|---|---|---|---|---|
| Location | Modification | Standard | 20 min | 40 min | 60 min | Control | Neutralization |
| N290 | Succinimide | 0.3 | 0.0 | 0.0 | 0.0 | 0.0 | 0.2 |
| N319 | Succinimide | 5.4 | 0.7 | 0.7 | 0.7 | 0.3 | 0.0 |
| N388 | Succinimide | 3.9 | 0.5 | 0.4 | 0.2 | 0.2 | 2.4 |
| N393 | Succinimide | 0.2 | 0.1 | 0.1 | 0.1 | 0.9 | 0.2 |
| Q3/Q13 | Deamidated | 0.0 | 0.5 | 1.1 | 2.8 | 0.1 | 0.1 |
| N56 | Deamidated | 0.7 | 6.0 | 7.2 | 10.7 | 1.0 | 1.6 |
| N280 | Deamidated | 0.0 | 10.0 | 14.2 | 21.7 | 0.3 | 0.7 |
| N290 | Deamidated | 0.9 | 1.6 | 2.2 | 3.5 | 0.1 | 0.1 |
| N365 | Deamidated | 0.0 | 9.4 | 13.6 | 23.7 | 0.0 | 0.0 |
| N388 | Deamidated | 3.0 | 21.0 | 22.8 | 28.9 | 0.8 | 2.6 |
| E1 | Pyro-Glu | 1.8 | 0.6 | 0.5 | 0.4 | 1.6 | 1.5 |
| W47/H50 | Oxidation | 0.0 | 0.5 | 1.0 | 1.0 | 0.1 | 0.1 |
| M85 | Oxidation | 0.1 | 4.5 | 3.4 | 2.6 | 1.8 | 1.2 |
| W111 | Oxidation | 0.7 | 0.1 | 0.1 | 0.0 | 0.0 | 0.2 |

TABLE 8
Post Translation Modifications in Light Chain (RPM LC-MS)

| | | Reference | | | | | Control w/o |
|---|---|---|---|---|---|---|---|
| Location | Modification | Standard | 20 min | 40 min | 60 min | Control | Neutralization |
| Q6 | Deamidated | 0.0 | 3.3 | 5.1 | 8.0 | 0.0 | 0.1 |
| N33 | Deamidated | 2.5 | 0.2 | 0.3 | 0.4 | 0.0 | 2.6 |
| N35 | Deamidated | 0.6 | 5.2 | 6.3 | 11.8 | 0.3 | 0.4 |
| N142 | Deamidated | 0.9 | 13.5 | 19.0 | 29.0 | 0.0 | 0.9 |
| N215 | Deamidated | 0.1 | 5.8 | 7.7 | 11.1 | 0.0 | 0.0 |
| M4 | Oxidation | 0.3 | 2.3 | 2.2 | 1.6 | 10.4¹ | 1.2 |

¹Control sample experienced minor induced oxidation due to sample handling during neutralization.

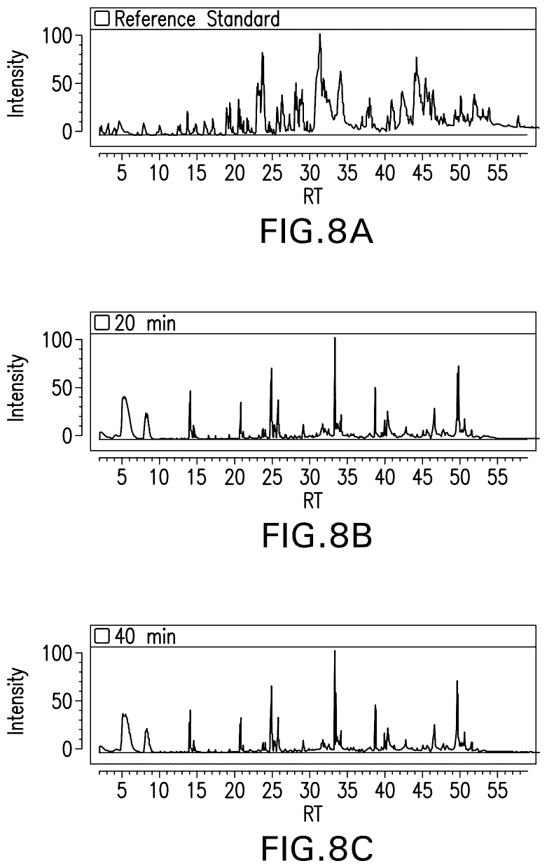

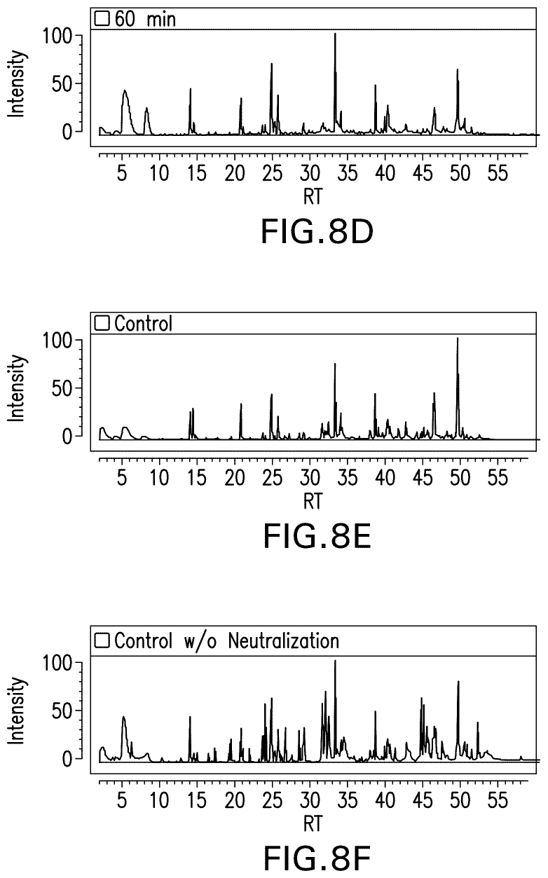

Decreases in sequence coverage (Table 9) and number of peaks in the total ion chromatograms by RPM after exposure to 1 M NaOH was observed relative to the controls which suggests that chemical degradation of the protein took place (FIGS. 8A-8F).

TABLE 9
Sequence Coverage for Product A Heavy
Chain and Light Chain (RPM LC-MS)

Sequence Coverage

| Sample | Heavy Chain | Light Chain |
|---|---|---|
| Reference Standard | 100.0% | 100.0% |
| 20 min | 83.6% | 95.9% |
| 40 min | 76.2% | 88.1% |
| 60 min | 62.2% | 72.6% |
| Control | 99.8% | 98.6% |
| Control w/o Neutralization | 99.2% | 96.8% |

Total organic carbon (TOC). Post GMP column preparation and blank run, the columns were flushed with WFI. The WFI samples from both AEX and CEX packed columns were analyzed using a TOC method. The method involves pre-treating the sample with dilute acid to remove carbonate carbon and then analyzing for total carbon using an instrument that utilizes a combustion system with an induction furnace coupled with a thermal conductivity detector (TCD) system and an IR detector system. This method is highly sensitive to residual carbon and is usually not performed on packed columns. The results for the WFI rinse are all below the limit of quantification (LOQ; 2 ppm) and align with the results obtained from the micro BCA, ELISA, and LC-MRM assays.

### Example 6: Blank Run and Sampling Design

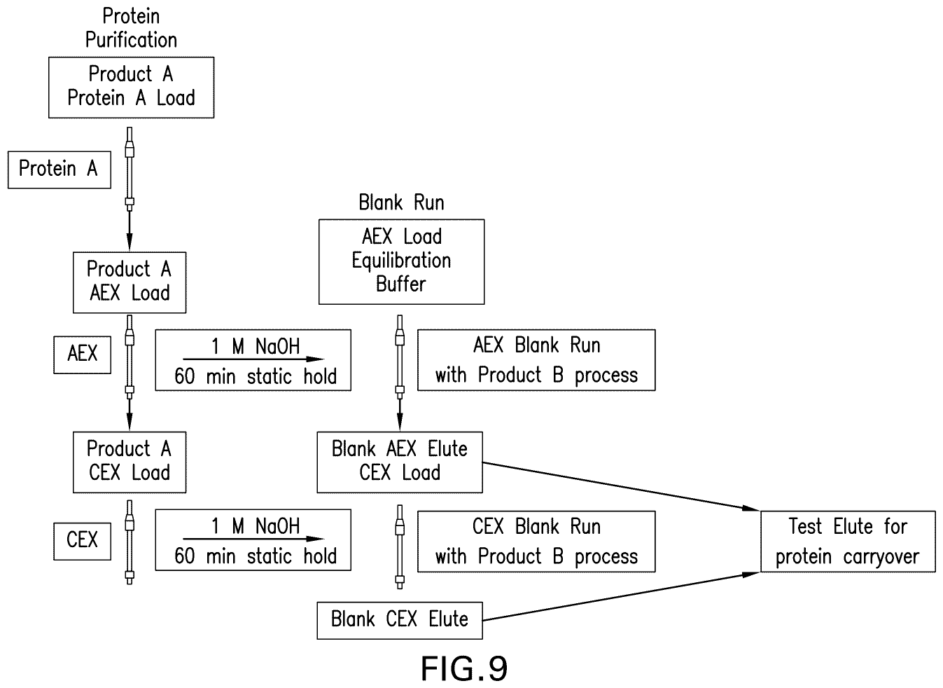

The NaOH concentration and contact time defined by the static hold degradation study was applied first to a lab-scale chromatography process to confirm the cleaning effectiveness. The lab-scale chromatography process used the same resin types, column heights, residence times, and buffers as a large-scale chromatography process. The anion exchange chromatography (AEX) resin, POROS 50HQ, and the cation exchange chromatography (CEX) resin, POROS 50HS, were then cleaned in place flushing first with a total of 5 column volumes of 1 M NaOH then a 60 min static hold. The resin was then subjected to a blank run using Product B AEX and CEX purification parameters. The blank runs followed Product B purification parameters without loading actual product on the resin. The AEX and CEX blank runs were linked by adjusting the AEX buffer eluate to serve as the load on the CEX column. Blank run eluate samples were collected and analyzed for Product A carryover and different analytical characterization testing. FIG. 9 outlines the blank run steps and sampling points. The blank run protocol developed at lab-scale was then applied to the pilot (non-GMP) and 2000 L GMP production scale AEX and CEX purification columns for both the pilot and GMP runs.

### Blank Elution Testing

A cleaning protocol was designed based on the degradation study outcome. Post use the columns were flushed with a total of 5 column volumes of 1 M NaOH followed by a 60 min static hold. Blank elution runs were then performed at small-scale. The small-scale purification, cleaning, and blank runs were completed a multiple of times to provide samples to quantitate the amount of degraded Product A present in the blank elution runs and to confirm inactivation and clearance. These samples were tested by ELISA, Micro BCA, and LC-MRM methods.

Micro BCA. Bicinchoninic acid (BCA) was used as the detection reagent for Cu⁺¹, which is formed when Cu⁺² is reduced by protein in an alkaline environment (Smith, P. K., et al. 1985 Anal Biochem 150:76-85 “Measurement of protein using bicinchoninic acid”). A purple-colored reaction product is formed by the chelation of two molecules of BCA with one cuprous ion (Cu⁺¹). This water-soluble complex exhibits a strong absorbance at 562 nm that is linear with increasing protein concentrations. The method utilized during development had a LOQ of 5 μg/mL and 6 μg/mL for the AEX and CEX eluate samples, respectively.

Multiple Reaction Monitoring-Liquid Chromatography (LC-MRM). Product A was spiked into phosphate-buffered saline (PBS) to generate a calibration curve (10 ng/mL-2000 ng/mL). Heavy labeled surrogate peptide DTLYITR (C13 and N15 at the last amino acid arginine) was spiked into the samples and calibrators at a constant level and used as an internal standard (IS). Samples and calibrators were denatured, reduced, alkylated, and digested by trypsin. The surrogate peptide DTLYITR, unique to Product A, was used as the analyte and the peak area ratio of analyte/internal standard (IS) was determined for quantification. The LOQ for this extended characterization method is 10 ng/mL. All results demonstrate that cleaning the column(s) with the above developed procedure sufficiently degrades and removes Product A (Table 10).

TABLE 10
Small Scale X (L) Blank Elution Runs
following 1M NaOH Sanitization

| Sample | Micro BCA¹ | ELISA² | LC-MRM³ |
|---|---|---|---|
| AEX Eluate Replicate 1 | <LOQ | <LOQ | <LOQ |
| AEX Eluent Replicate 2 | <LOQ | <LOQ | <LOQ |
| AEX Eluent Replicate 3 | <LOQ | <LOQ | <LOQ |
| CEX Eluate Replicate 1 | <LOQ | <LOQ | <LOQ |
| CEX Eluate Replicate 2 | <LOQ | <LOQ | <LOQ |
| CEX Eluate Replicate 3 | <LOQ | <LOQ | <LOQ |

¹Micro BCA LOQ for CEX Eluate = 6 μg/mL, LOQ for AEX Eluate = 5 μg/mL. These values represent the LOQ of the method used during development.
²ELISA LOQ = 3 ng/mL. This value represents the LOQ of the method used during development.
³LC-MRM LOQ = 10 ng/mL. This assay is used for extended characterization.

When the cleaning procedures were scaled-up to the pilot- and GMP-scale, blank run samples were tested with the Micro BCA and ELISA methods. As shown in Table 11 and Table 12, the results confirmed that Product A was effectively inactivated and removed across all scales of blank runs for MRR.

TABLE 11
Pilot Scale Blank Elution Runs following 1M NaOH Sanitization

| Sample | Micro BCA | Residual ELISA | LC-MRM |
|---|---|---|---|
| AEX Eluate | <LOQ | <LOQ | <LOQ |
| CEX Eluate | <LOQ | <LOQ | <LOQ |

¹Micro BCA LOQ for CEX Eluate = 6 μg/mL, LOQ for AEX Eluate = 5 μg/mL.
²ELISA LOQ = 3 ng/mL for CEX and AEX Eluate.
³LC-MRM LOQ = 10 ng/mL.

TABLE 12
GMP Scale Blank Elution Runs following 1M NaOH Sanitization

| Sample | Micro BCA | Residual ELISA | LC-MRM |
|---|---|---|---|
| AEX Eluate | <LOQ | <LOQ | <LOQ |
| CEX Eluate | <LOQ | <LOQ | <LOQ |

¹Micro BCA LOQ for CEX Eluate = 3 μg/mL, LOQ for AEX Eluate = 5 μg/mL.
²ELISA LOQ for CEX Eluate = 5 ng/mL, LOQ for AEX Eluate = 5 ng/mL.
³LC-MRM LOQ = 10 ng/mL.

### Acceptance Criteria for Product a Carryover

Resin cleaning protocols are often designed to expose proteins to extreme pHs for an extended period. Under these conditions, proteins are known to degrade and denature (Kendrick K, et al. J. Validation Technol., 2009, 15 (3), 69-77 “Analysis of Degradation Properties of Biopharmaceutical Active Ingredients as Caused by Various Process Cleaning Agents and Temperature”). As shown above in Table 6, Product A is completely inactivated by 1 M NaOH hold as measured by the product-specific ELISA activity assay showing the results were all below the limit of quantitation of the assay (3 ng/mL established for Product A) as well as several other characterization assays. Once it is confirmed that the NaOH cleaning procedure degraded Product A into fragments and no active protein remains on the cleaned resins, the acceptance limits for the total protein fragment carryover for multiproduct resin reuse need to be established to ensure the product safety. For establishing the Product A acceptance limits, a scientific rationale was followed based on the use of gelatin as a reference impurity (Sharnez R et al. 2013 Journal of Validation Technology, 2013, 19 (1) “Acceptance limits for inactivated product based on Gelatin as a reference impurity”). The acceptance limit of total protein carryover is 650 μg/dose, determined by clinical safety data of gelatin impurity in LUPRON DEPOT, which is a marketed drug that is dosed monthly. To determine the total protein carryover for Product A, the number of doses in a drug substance production batch was calculated following the steps described in Table 13.

TABLE 13
Number of Doses in a Clinical Batch of Product A

| Parameters | Formula |
|---|---|
| Bioreactor volume (B, L) | — |
| Titer (P, g/L) | — |
| Approximate DS yield ratio (Y) | — |
| Projected maximum clinical dose of Product B (D, g/dose) | — |
| Product B mass harvested from bioreactor (H, g) | B × P |
| Mass in DS (M, g) | H × Y |
| Number of doses per batch (N) | M/D |

The acceptable total protein carryover in the final elution buffer of the blank buffer run was then calculated following the steps in Table 14.

TABLE 14
Acceptance Limit of Protein Carryover
in Final Blank Elution Sample

| Parameters | Formula |
|---|---|
| Column diameter (d, cm) | — |
| Column bed height (h, cm) | — |
| Blank elution volume (column volume | — |
| number n) | |
| Total protein carryover allowed in a dose | — |
| of Product B (a, μg/dose) | |
| Number of doses per batch (N), from Table 13 | — |
| Column volume (v, L) | h × p × (0.5 × d)²/1000 |
| Total blank elution volume (b, L) | n × v |
| Total protein carryover allowed in a | N × a/1000 |
| clinical batch (t, mg) | |
| Protein carryover limit in final blank | t/b |
| elution sample (μg/mL) | |

### Example 7: Execution of an MRR GMP Process for Production of a Biopharmaceutical

A failure modes and effects analysis (FMEA) based risk assessment was executed by a GMP manufacture team and a site quality assurance team with the input from a technical development team for risk management. Risks associated with product cross contamination, resin reuse, and process performance were analyzed and categorized, and appropriate risk controls were put in place prior to the GMP campaign. Typical GMP campaign activities, such as equipment, flow path clean verification, endotoxin and bioburden testing were also executed following standard procedures.

Across scales, sites, and campaigns there was no discernable trends observed between multiproduct and single product resin reuse. Selected process performance results of the GMP run were compared to the pilot-scale and development runs, which included the yield of AEX and CEX unit operation in the Product B manufacturing process and Product B process intermediate and drug substance quality data (data not shown here). The results were consistent across scales, sites, and campaigns indicating overall process performance and resulting product quality was not impacted by multiproduct resin use.

In addition, standard criteria were put in place for regular cleaning validation testing including endotoxin and bioburden. A viral clearance study was also performed using the Product B GMP campaign process intermediates. Overall clearance capacity using the two model viruses, X-MuLV and MMV, was achieved similarly to that of the Product A process and other typical antibody-based processes (data not shown).

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method to prepare a chromatography material used in the purification of a first polypeptide for reuse in the purification of a second polypeptide, the method comprising the steps of:
a) obtaining the chromatography material used in the purification of the first polypeptide, optionally wherein the first polypeptide has been eluted from the chromatography material;
b) passing one or more material volumes of regeneration buffer through the chromatography material, wherein the regeneration buffer comprises from about 0.05 M to about 2.0 M NaOH and a pH from about pH 12.0 to about pH 14.0;
c) incubating the chromatography material from about 0° C. to about 32° C. for at least from about 10 minutes to at least about 180 minutes in the presence of at least one of the one or more material volumes of regeneration buffer;
d) adding one or more material volumes of elution buffer to the chromatography material, wherein the elution buffer is formulated for use with the chromatography material and the first polypeptide;
e) optionally collecting the one or more material volumes of elution buffer from the chromatography material eluate, and optionally testing the one or more material volumes for the presence of the first polypeptide; and
f) equilibrating the chromatography material for reuse with the second polypeptide.

2. The method of claim 1, wherein the first and second polypeptides are the same or are different.

3. (canceled)

4. The method of claim 1, wherein the first and second polypeptides are selected from the group consisting of: an enzyme; a hormone; a fusion protein; an Fc-containing protein; an immunoconjugate; a cytokine; and an antibody or antigen binding fragment thereof;
optionally wherein the antibody is selected from the group consisting of: a monoclonal antibody; a chimeric antibody; a humanized antibody; a human antibody; and a multispecific antibody:
optionally wherein the multispecific antibody is a bispecific antibody or a trispecific antibody; and
optionally wherein the antigen binding fragment thereof is selected from the group consisting of: a Fab fragment; a Fab′ fragment; a F(ab′)2 fragment; an scFv; a di-scFv; a bi-scFv; a tandem (di, tri) scFv; an Fv; a sdAb; a tri-functional antibody; a BiTE; a diabody; and a triabody.

5-7. (canceled)

8. The method of claim 1, wherein the first polypeptide has been eluted or has not been eluted from the chromatography material in step (a).

9. (canceled)

10. The method of claim 1, wherein the chromatography material comprises first and second chromatography materials; optionally wherein the first and second chromatography materials are operated discontinuously or continuously in series.

11-12. (canceled)

13. The method of claim 1, wherein one, two, three, four, five, or more than five material volumes of regeneration buffer is passed through the chromatography material.

14-18. (canceled)

19. The method of claim 1, wherein the regeneration buffer comprises about 0.05, 0.1, 0.5, 1.0, 1.5, or 2.0 M NaOH and a pH at about pH 13.0.

20-24. (canceled)

25. The method of claim 1, wherein the chromatography material is incubated at from about 20° C. to about 22° C. for at least about 10, 20, 40, 60, 120, or 180 minutes in the presence of at least one of the one or more material volumes of regeneration buffer.

26-30. (canceled)

31. The method of claim 1, wherein the chromatography material is selected from the group consisting of: an affinity chromatography material; an ion exchange chromatography material; a monolith chromatography material; a hydrophobic interaction chromatography material; and a mixed mode chromatography material;
optionally wherein the affinity chromatography material is a protein A affinity chromatography material;
optionally wherein the protein A affinity chromatography material is selected from the group consisting of: MabSelect; MabSelect SuRe; and MabSelect SuRe LX;
optionally wherein the ion exchange chromatography material is an anion exchange chromatography (AEX) material;
optionally wherein the AEX material is POROS 50HQ;
optionally wherein the ion exchange chromatography material is a cation exchange chromatography (CEX) material; and
optionally wherein the CEX material is Poros 50HS.

32-37. (canceled)

38. The method of claim 1, wherein the elution buffer comprises acetic acid and/or from about 0.01 M to about 0.5 M sodium acetate at a pH of from about pH 2.5 to about pH 5.0.

39. The method of claim 1, wherein optionally testing the one or more material volumes of elution buffer for the presence of the first polypeptide comprises one or more analytical assay methods selected from the group consisting of: non-reduced sodium dodecyl sulfate-polyacrylamide gel electrophoresis (SDS PAGE); ultra-performance liquid chromatography-size exclusion chromatography (UPLC-SEC); intact mass liquid chromatography-mass spectrometry (IM LC-MS); reduced peptide mapping (RPM); multiple reaction monitoring-liquid chromatography (LC-MRM); micro BCA; residual binding enzyme-linked immunosorbent assay (ELISA); and total organic carbon (TOC).

40. The method of claim 39, wherein the first polypeptide is present in the one or more material volumes of elution buffer below a level of quantitation (LOQ) for the one or more analytical assay methods selected.

41. The method of claim 39, wherein the first polypeptide is present in the one or more material volumes of elution buffer as one or more protein fragments, and wherein a total amount of polypeptide fragments are below an acceptable limit for a reference purity; optionally wherein the reference impurity is gelatin; and optionally wherein the acceptable limit is 650 μg/dose.

42-43. (canceled)

44. The method of claim 1, wherein the chromatography material is linked continuously or discontinuously to one or more preceding or subsequent chromatography materials.

45. The method of claim 1, wherein the reused chromatography material is used to manufacture the second polypeptide at large scale, optionally wherein the large scale is a GMP scale.

46. A method to prepare chromatography material used in the purification of a first polypeptide for reuse in the purification of a second polypeptide, the method comprising the steps of:
a) obtaining the chromatography material used in the purification of the first polypeptide, optionally wherein the first polypeptide has been eluted from the chromatography material;
b) passing one or more material volumes of regeneration buffer through the chromatography material, wherein the regeneration buffer comprises about 1.0 M NaOH at a pH of about pH 13.0;
c) incubating the chromatography material at from about 20° C. to about 22° C. for about 20 minutes in the presence of at least one of the one or more material volumes of regeneration buffer;
d) adding one or more material volumes of elution buffer to the chromatography material, wherein the elution buffer comprises from about 0.01 M to about 0.5 M sodium acetate at a pH of from about pH 2.5 to about pH 5.0;
e) optionally collecting the one or more material volumes of elution buffer from the chromatography material eluate, and optionally testing the one or more material volumes for the presence of the first polypeptide; and
f) equilibrating the chromatography material for reuse with the second polypeptide.

47. The method of claim 46, wherein the first and second polypeptides have acceptable daily exposure or permitted daily exposure limits more than 10 μg/day; optionally wherein the first and second polypeptides have No-Observed-Effect-Level for systemic toxicity of ≥300 mg/kg/week.

48. (canceled)

49. A polypeptide produced or obtainable by the method of claim 1.

50. (canceled)

51. The polypeptide of claim 49, wherein the polypeptide is selected from the group consisting of: an enzyme; a hormone; a fusion protein, an Fc-containing protein; an immunoconjugate; a cytokine; and an antibody or antigen binding fragment thereof;
optionally wherein the antibody is selected from the group consisting of: a monoclonal antibody; a chimeric antibody; a humanized antibody; a human antibody; and a multispecific antibody;
optionally wherein the multispecific antibody is a bispecific antibody or a trispecific antibody; and
optionally wherein the antigen binding fragment thereof is selected from the group consisting of: a Fab fragment; a Fab′ fragment; a F(ab′)2 fragment; an scFv; a di-scFv; a bi-scFv; a tandem (di, tri) scFv; an Fv; an sdAb; a tri-functional antibody; a BiTE; a diabody; and a triabody.

52-55. (canceled)

56. A composition comprising a therapeutically effective amount of the polypeptide of claim 49, and a pharmaceutically acceptable carrier.
